(19) United States
(12) Patent Application Publication
(10) Pub. No.: US 2024/0265529 A1
(43) Pub. Date: Aug. 8, 2024

(54) AUTOMATIC DETECTION OF ANATOMICAL LANDMARKS AND EXTRACTION OF ANATOMICAL PARAMETERS AND USE OF THE TECHNOLOGY FOR SURGERY PLANNING

(71) Applicants: AliAsghar MohammadiNasrabadi, Waterloo (CA); John Joseph McPhee, Waterloo (CA)

(72) Inventors: AliAsghar MohammadiNasrabadi, Waterloo (CA); John Joseph McPhee, Waterloo (CA)

(21) Appl. No.: 18/435,906

(22) Filed: Feb. 7, 2024

Related U.S. Application Data
(60) Provisional application No. 63/443,937, filed on Feb. 7, 2023.

(51) Int. Cl. G06T 7/00; G06T 7/60

(52) U.S. Cl. CPC G06T 7/0012; G06T 7/60; G06T 2207/10081; G06T 2207/10088; G06T 2207/10116; G06T 2207/10132; G06T 2207/30012

(57) ABSTRACT

The measurement of anatomical parameters is an essential pre-operative, intra-operative, and post-operative procedure for surgery. Traditionally, surgeons manually annotate anatomical landmarks to extract these parameters from medical images. However, our invention focuses on the automatic detection of anatomical landmarks to extract these parameters and the use of this technology for patient categorization and surgery planning. To accomplish this, a deep learning model can be trained with various datasets, such as lateral X-rays, Anterior Posterior (AP) X-rays, Computed tomography (CT-scans), Magnetic resonance imaging (MRI), and Ultrasound images. A physics-informed approach is utilized to enhance the performance of the deep learning model by defining the geometric relations between landmarks during training. Furthermore, a computing device can be used to receive a medical image of a patient as input, and surgeons can specify which parameters they would like to detect. The device will then activate the corresponding model, perform various image processing tasks, and compute the locations of the specified anatomical landmarks in order to detect the parameters. The resulting measurements can then be used to classify patients according to their anatomical conditions. An interactive graphical user interface (GUI) can also be provided, allowing surgeons to relocate any of the detected landmarks to meet their needs (i.e., to correct any possible errors in landmarks). The model will then be retrained accordingly to improve future predictions. Additionally, the computing device can provide surgical guidance to surgeons based on the classification it performs.

### CROSS-REFERENCE TO RELATED APPLICATIONS

This application claims the benefit of U.S. Provisional Application No. 63/443,937 filed on Feb. 7, 2023, and entitled AUTOMATIC DETECTION OF ANATOMICAL LANDMARKS AND EXTRACTION OF ANATOMICAL PARAMETERS AND USE OF THE TECHNOLOGY FOR SURGERY PLANNING, the entirety of which is incorporated herein by reference.

### FIELD OF THE INVENTION

The present invention generally relates to automatic detection of anatomical landmarks and extraction of anatomical parameters from medical images and use of the technology to support surgical planning.

### BACKGROUND

Surgeons require accurate and consistent measurements of anatomical parameters (e.g., spinopelvic parameters, alignment, leg length, joint gap, etc.) before, during, and after surgery in order to plan, perform, and evaluate their operations. The traditional method of extracting these parameters is manual labeling of images, which is time-consuming and can result in inconsistent annotations between different surgeons. Utilizing a reliable, automated extraction of these parameters can not only address the drawbacks of manual labeling, but can also provide a platform for patient classification and surgical planning.

To provide a means for automatic extraction of anatomical landmarks, several intelligent systems have been proposed, including heatmap-based regression and segmentation approaches. Although these are the most common methods used for anatomical landmark detection, such as the approaches described in Farrantelli et al (US20210118134) and Bronkalla (US20180068067), they have some inherent drawbacks, such as overlapping signals, quantization error, mis-detection due to low quality of X-ray images, and high computational cost. Semi-automated systems, like Bronkalla (US20180068067), still need user intervention for the detection, thereby retaining some of the manual labeling drawbacks. Moreover, Bronkalla (US20180068067) is limited to the spine and cannot handle cases where an external obstacle (e.g., X-ray safety plates) partially obstructs the view of the X-ray. Additionally, the potential of this technology to improve surgical planning and operation outcome has yet to be clarified.

What is therefore needed is a new approach for the detection of anatomical landmarks and extraction of anatomical parameters from medical images which addresses at least some of these limitations in the prior art.

### SUMMARY

Surgeons measure anatomical parameters to plan and evaluate their surgery, and the automatic extraction of these parameters saves time, provides consistent measurements, and avoids human error compared to manual parameter extraction. This technology enables efficient and accurate patient categorization and surgical planning.

The present disclosure provides a system and method for the automatic detection of anatomical landmarks and extraction of anatomical parameters from medical images, and its utilization for patient categorization and support of surgical planning. To achieve this, a deep learning model can be trained with various datasets, such as lateral X-rays, AP X-rays, CT-scans, MRI, and Ultrasound images. Additionally, the performance of the model may be further improved through the implementation of a physics-informed approach, which introduces the geometric relation between different landmarks to the model to provide a global understanding of the images.

For each anatomical parameter, certain anatomical landmarks must be extracted. A computing device is utilized to receive a medical image as an input, and surgeons can indicate the desired parameters to be identified. The computing device then activates the corresponding trained model and performs various image processing tasks to detect the location of the required anatomical landmarks to measure the specified parameters. This measured data may be used to classify patients in regard to different anatomical conditions. The computing device may also be programmed to keep track of the detected parameters, and an interactive GUI can be employed as a possible embodiment, allowing surgeons to relocate any of the detected landmarks to meet their requirements (i.e., to correct any possible errors in detected landmarks). The computing device will keep a record of these alterations, recording the new annotation that can be utilized as an augmented dataset for the model to be retrained, thus improving its future predictions. The measured parameters may be utilized to categorize patients and provide pre-operative, intra-operative, and post-operative surgical guidance. Additionally, the concept of landmarks as objects is introduced, allowing the technology to be utilized for any kind of medical images. The method can be applied to extract parameters from X-ray images of different views (i.e., lateral, AP), MRI, Ultrasonic, and CT-scan images to detect the necessary landmarks and extract the desired parameters.

One possible embodiment of the technology is to use it for the extraction of anatomical parameters from lateral X-ray images. A user-friendly graphical interface (GUI) has been developed to facilitate this process, which only requires the users to upload the desired image. The most significant parameters to be evaluated in lateral X-ray images by surgeons are the Sacral Slope (SS), Pelvic Tilt (PT), Pelvic Incidence (PI), Lumbar Lordosis (LL), and Sagittal Vertical Axis (SVA). These parameters can be extracted from the images by identifying certain anatomical landmarks. The extracted parameters are then utilized to categorize patients into four distinct groups based on spinal stiffness. This categorization helps hip surgeons to make informed decisions and select the most appropriate surgical approach for an optimal outcome.

An additional embodiment of the present invention encompasses its application in extracting anatomical parameters from anteroposterior (AP) X-ray images. This technology is adept at classifying patients according to the severity of scoliosis conditions. Furthermore, the extraction of anatomical parameters from AP images is instrumental for surgeons in assessing pelvic tilt and overall spinal alignment in the AP view, thereby enhancing the precision and effectiveness of surgical evaluations and planning. The alignment of the vertebrae is a primary concern in scoliosis. The proposed technology can assess deviations from the normal vertebral column alignment, particularly in the coronal plane using the extracted necessary landmarks automatically.

As an illustrative example, to assess scoliosis condition, the Cobb angle may be measured. This is a standard method for quantifying the degree of spinal curvature. However, it is one of several methods for measuring and assessing scoliosis, which may also be used.

In the presented technology, lines parallel to the top of the uppermost tilted vertebra and the bottom of the lowest tilted vertebra in the curve are identified, then drawing perpendicular lines to these which intersect. The angle where these perpendicular lines intersect is the Cobb angle. For the pelvis in the coronal plane, the focus is often on pelvic obliquity. Pelvic obliquity refers to the tilt of the pelvis when one hip is higher than the other. Pelvic obliquity can be measured on an AP X-ray by drawing horizontal lines at the tops of the iliac crests or the pelvic brims. The difference in height between these lines indicates the degree of obliquity. The presented technology can automatically detect required landmarks and evaluate the pelvic obliquity as described below, but the approach can also be used for automatically detecting required landmarks for evaluating various other anatomical parameters for different conditions or diseases.

Surgeons should spend an average of 3-5 minutes to annotate each image, depending on the number of landmarks they need to locate in order to extract certain parameters. Assuming a surgeon visits 10 patients a day, and annotates two images (e.g., X-ray images of sitting and standing postures) for each patient, it would take them between 60-100 minutes per day to complete the task—a task that can now be done automatically. The value of this invention is evident when we consider the current waiting time for surgeons, which can be up to 6 months or even a year, and take into account the number of neurosurgeons and spine surgeons in North America; there are more than 30,000 orthopedic and neurosurgeons working in this region. With this in mind, our invention can save 30,000-50,000 hours' worth of work from surgeons every day. Consequently, this invention can significantly reduce the amount of time patients must wait to see a surgeon.

The technical details of the invention are provided in the following paragraphs and drawings provided herewith. The invention, its characteristics, objectives, and advantages are described in such a way as to be understood by those skilled in the art.

### BRIEF DESCRIPTION OF THE DRAWINGS

FIG. 1 shows a drawing that represents a lateral view of a medical image and introduces different anatomical parameters and corresponding anatomical landmarks.

FIG. 2 shows one type of medical image (a lateral view X-ray) and the preparation of the image for training a deep learning model.

FIG. 3A is a schematic block diagram illustrating how a computer system is used to develop and train a deep learning model and how this model can be used to extract desired anatomical parameters in accordance with an embodiment.

FIG. 3B is an illustrative lateral x-ray image, considering both femoral heads to calculate the center of rotation for the pelvis in accordance with an embodiment.

FIG. 3C shows an illustration of manual labeling of landmarks and specifying bounding boxes in accordance with an embodiment.

FIG. 3D shows a LanDet utilizing a dense detection network denoted as DN that is trained with a multi-task loss in accordance with an embodiment.

FIG. 3E shows illustrative ICC metric results for the evaluation of model reliability by comparing the extracted results from the model with manual annotation by a number of participating surgeons.

FIG. 3F shows illustrative examples of challenging cases successfully addressed by the model in the datasets: A) Partially cut-off images, B) Images with obstacles in the hip region, C) Images from patients with hip implants, D) Images from patients with spinal implants.

FIG. 3G shows the advantage of applying physics-informed constraints, which improved the accuracy of localization of some similar and adjacent landmarks.

FIG. 4 shows a possible output of the model (anatomical parameters) of a lateral view X-ray image presented to the user comparing with ground truth manual annotation.

FIG. 5 illustrates a potential embodiment of the technology to extract spinopelvic parameters for both sitting and standing postures, comparing the parameters, and classifying patients based on various anatomical parameters and spine stiffnesses.

FIG. 6 is a flow diagram illustrating how a user can utilize the technology by importing a medical image. It outlines the different processing steps necessary to automatically extract the desired anatomical parameters and subsequently categorize patients based on the detected parameters.

FIG. 7 illustrates a representative output generated by the model, depicting anatomical landmarks in the AP view derived from AP X-ray images.

### DETAILED DESCRIPTION

A detailed technical description of the present invention, including a method for automatic anatomical landmark detection and extraction of anatomical parameters using a physics-informed deep learning approach, is provided with reference to the attached illustrations and diagrams.

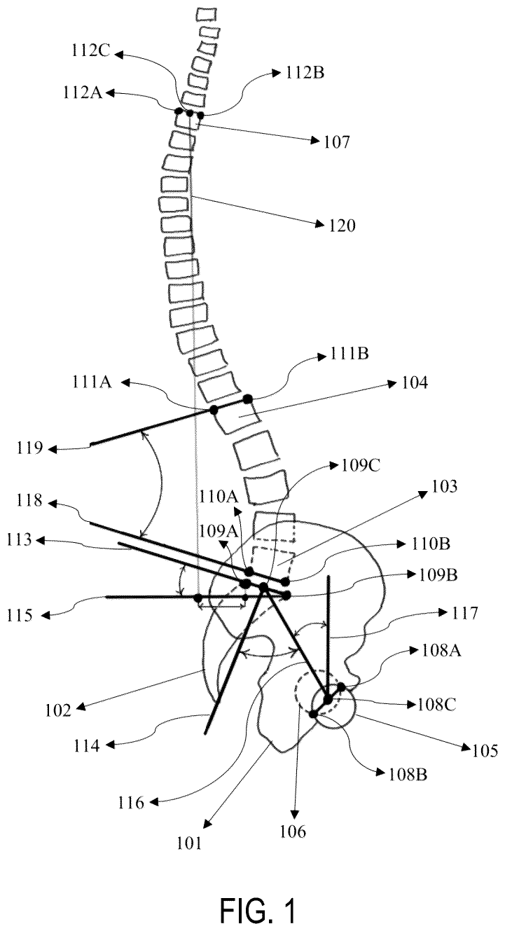

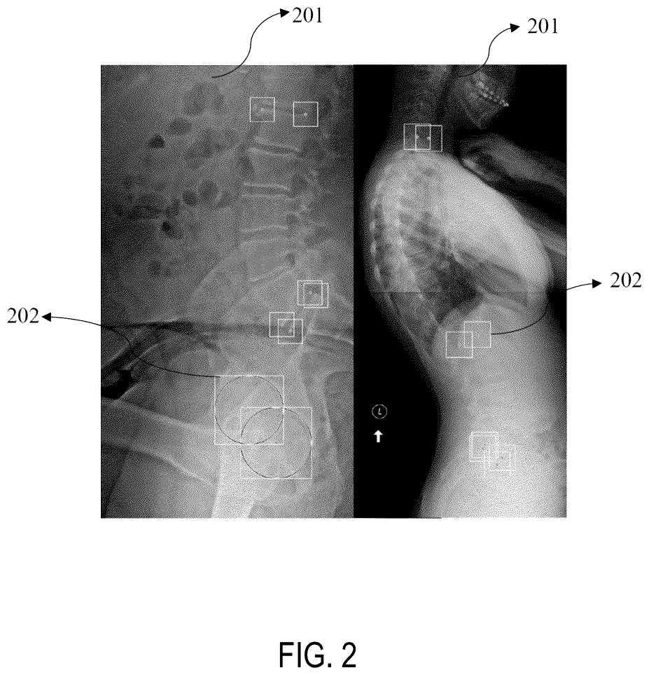

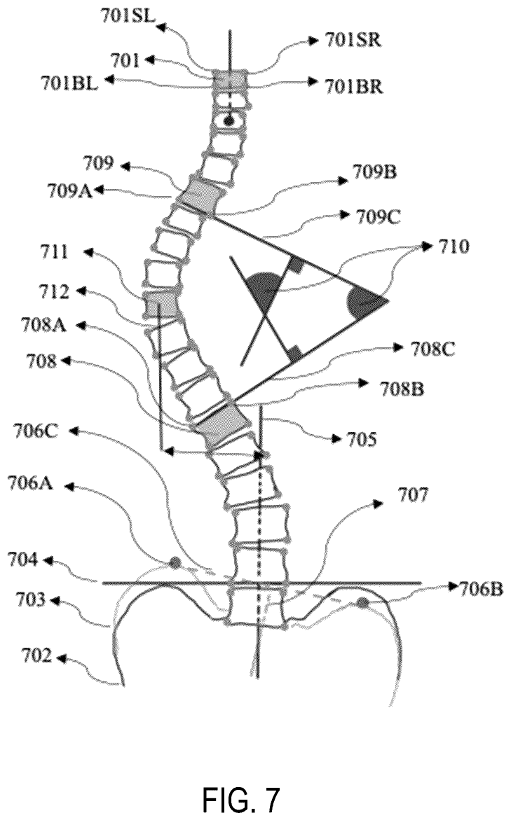

Beginning with FIG. 1, a lateral view drawing indicates the anatomical parameters and required landmarks to be detected to measure the parameters from “medical images”. The term “medical images” refers to any images taken from patients including but not limited to X-rays (lateral view shown in FIG. 2, and AP view shown in FIG. 7), MRI, CT-scan, and Ultrasound and including but not limited to images from full body, spine, pelvis, and spine with pelvis. In the schematic lateral image 1, part 101 represents the pelvis, part 102 represents the sacrum, 103 represents the lowest lumbar spine vertebra (L5), part 104 represents the last upper vertebra in the lumbar spine region (L1), parts 105 and 106 represent femoral heads, and part 107 represents the lowest vertebra of the cervical part of the spine.

There are some anatomical parameters that should be defined here. Sacrum Slope (SS) refers to the slope of the sacrum 102 and defined as the angle between the tangent line 113 to the upper endplate of sacrum (connecting line between posterior 109A and anterior 109B corners of sacral upper endplate) and the horizontal reference line 115. The intersection points 108A and 108B of femoral heads 105 and 106, are used to find the imaginary center point 108C which henceforth is referred to as the “femoral head”. Pelvic Tilt (PT) refers to the tilting angle of the pelvis and is defined as the angle between the connecting line of sacrum midpoint 109C and femoral head 108C, and the reference vertical line 117. Pelvic Incidence (PI) is defined as the angle between the perpendicular line 114 to the line 113 in the midpoint of sacrum plate 109C and the connecting line 116 of sacrum midpoint 109C and femoral head 108C. Lumbar Lordosis (LL) represents the curvature of the lumbar spine and is defined as the angle between the tangent line 118 to the L5 endplate (connecting line between the posterior 110A and anterior 110B corners of L5 upper endplate) and the tangent line 119 to the L1 endplate (connecting line between the posterior 111A and anterior 111B corners of L1 upper endplate). Sagittal Vertical Axis (SVA) is used as a spine alignment parameter and has been defined as the distance from the plumb line 120 from the center 112C of the C7 vertebra 107 upper end plate (the midpoint of the connecting line between the posterior 112A and anterior 112B of the upper endplate of C7 vertebra 107's corners) to the posterior corner 109A of the upper sacral endplate. The defined parameters SS, PT, PI, LL, and SVA are henceforth referred to as the “anatomical parameters” that are used to evaluate the spinal condition before, during, and after the sugary. The term “anatomical parameters” is not limited to the parameters measured In the lateral view X-ray images and includes but is not limited to any anatomical parameter that can be measured from lateral and AP X-ray, CT-scans, MRI, and Ultrasonic images. To extract the anatomical parameters one possible approach is to detect and locate certain points, which in the lateral view X-ray image described here are: 108A, 108B, 109A, 109B, 110A, 110B, 111A, 111B, 112A, and 112B, henceforth are referred to as the “anatomical landmarks”.

Still referring to FIG. 1A, the assessment and prediction of the geometric characteristics of the spinopelvic complex have garnered significant interest among both the clinical and research communities. Radiological examination of the spine and pelvis plays a crucial role in both surgical and non-surgical treatments of spinal disorders. Understanding the sagittal balance of the spine and pelvis, which entails the interplay between spine and pelvic measures, is crucial for maintaining postural equilibrium. Initially, measurements of sagittal balance were conducted manually using conventional radiographs and later assisted by computer-based tools. However, the inherent limitations of radiographic imaging and subjectivity in manual measurements introduced errors.

To address these challenges, machine learning techniques, a subset of artificial intelligence (AI), were employed, allowing computer models to recognize patterns in data. With the advancement of deep learning (DL), a specialized branch of machine learning that emulates the information processing of neural systems, performance in automated image analysis significantly improved. DL methods excel in learning optimal features and feature compositions without human-designed feature extraction. Consequently, DL has found extensive application in various domains, including radiology, musculoskeletal radiology, and spinal disorders.

The concept of sagittal spinopelvic balance has gained widespread recognition among radiologists and spine professionals, as it is essential for understanding the etiopathogenesis of spinal deformities and selecting appropriate treatment options. Evaluating sagittal balance typically involves radiographic measurements of geometric relationships among specific anatomical landmarks in sagittal X-ray images. Measures such as Sacral Slope (SS), Pelvic Tilt (PT), Pelvic Incidence (PI), Lumbar Lordosis (LL), and Sagittal Vertical Axis (SVA) are commonly used to assess sagittal balance. However, manual measurements can be subjective, time-consuming, and prone to inaccuracies due to the complexity of accurately identifying anatomical landmarks. To overcome these challenges, various computer-assisted tools and software have been developed, but they still rely on observer input.

Referring to FIG. 2, shown is a preparation process on medical image 201 by creating bounding boxes 202, and the center of each bounding box represents each anatomical landmark. To assess sagittal spinopelvic measures, two primary methods are generally employed. The first approach involves landmark detection, often complemented by preliminary structure detection, followed by direct geometric measurements using the identified landmarks. The second approach utilizes structure segmentation, followed by landmark and/or inclination detection from the segmented structures. Notably, convolutional neural networks (CNNs) were frequently used, where input images underwent convolutional and pooling layers for feature extraction and fully connected layers for feature classification. However, the trend is now shifting towards segmentation with CNNs, incorporating encoder-decoder architectures.

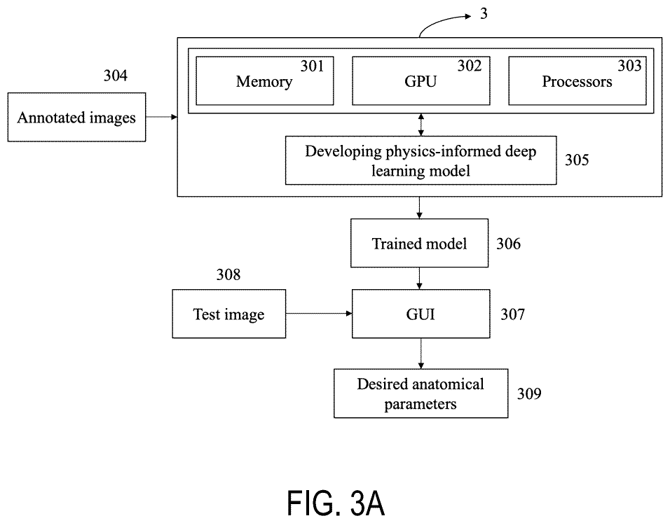

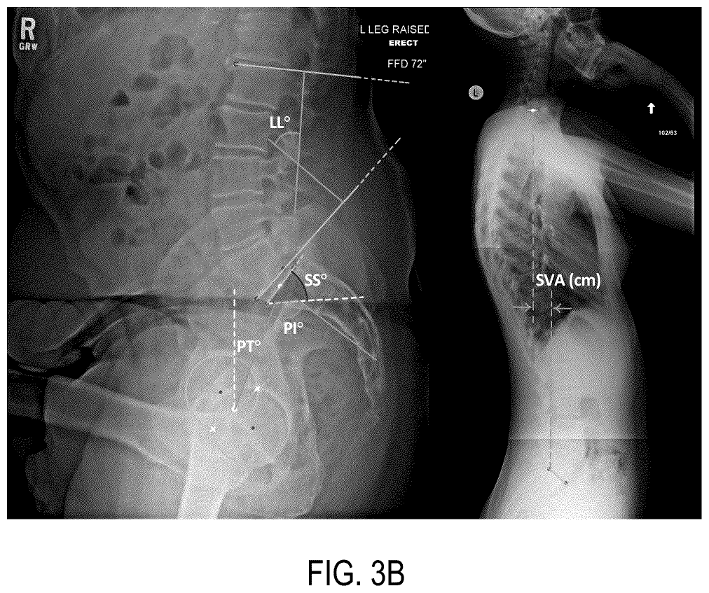

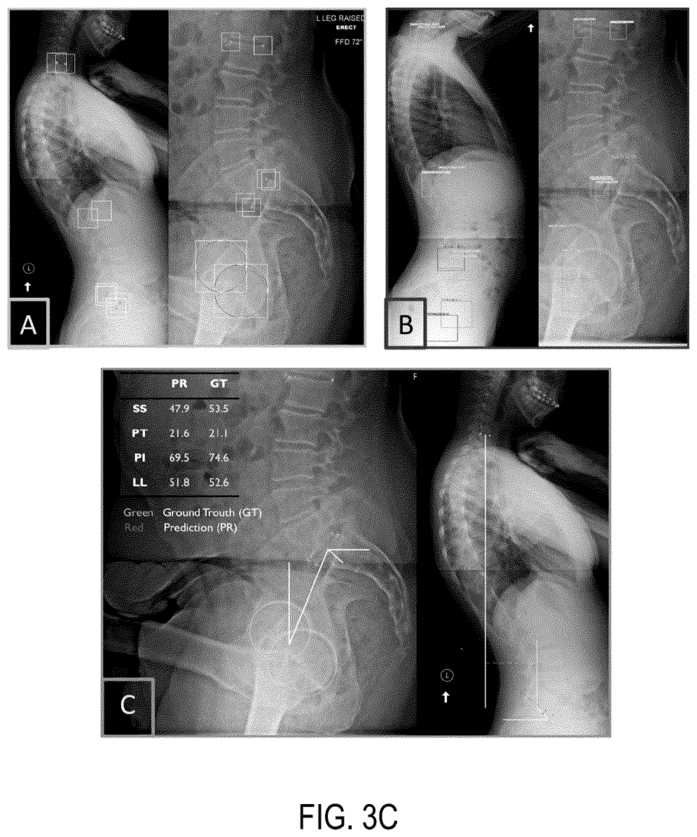

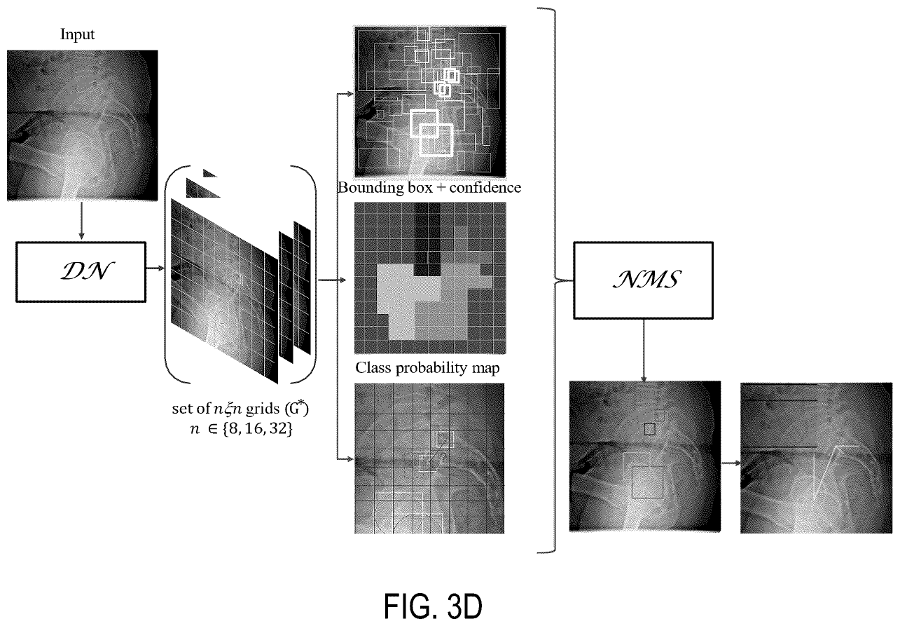

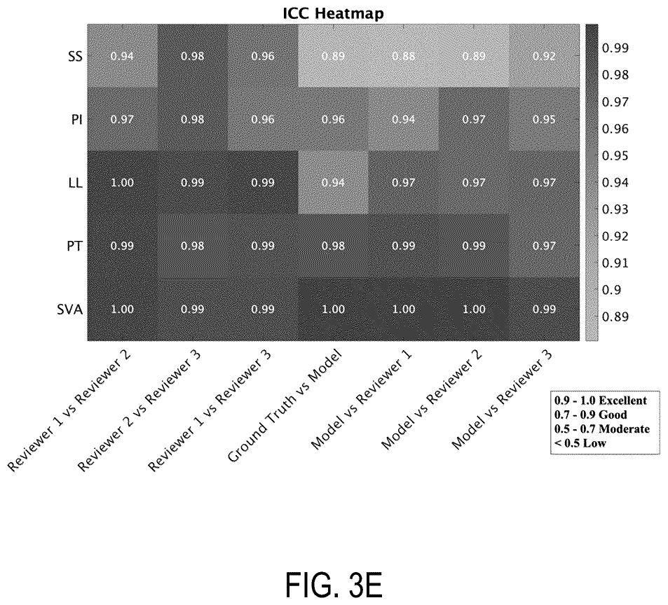

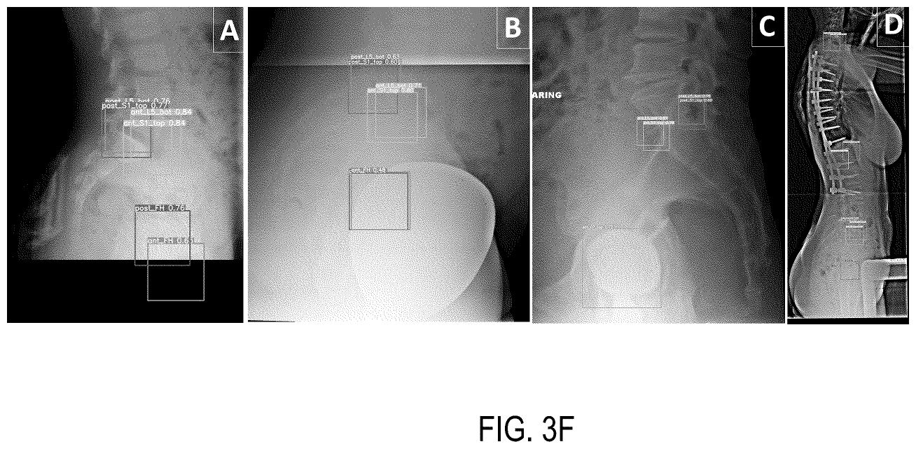

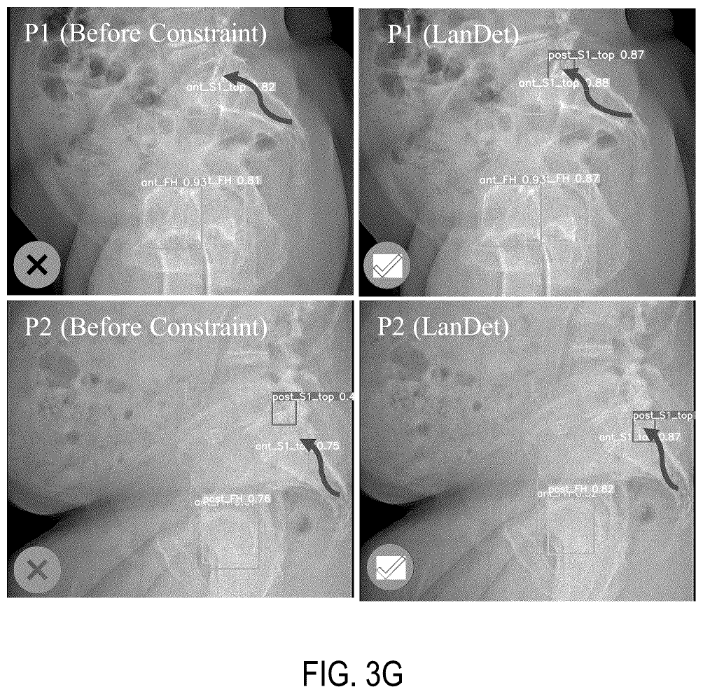

Now referring to FIG. 3A, shown is a computer system 3 including but not limited to memory, GPUs, and processors, is used to feed the annotated images 304 to the deep learning model 305 and is used to train the model and provide optimized hyper-parameters of the trained model 306. The trained model 306 then can be used as the source for anatomical landmark detection and anatomical parameter extraction. The deep learning model 305 is an object detection model which has been improved by a physics-informed approach in which the geometrical relations between anatomical landmarks are introduced to the model as the anatomical parameters to give a global understanding of images to the model. By utilizing the introduced method (i.e., geometric relations as constraints in the loss function of the model) or any similar approach, including but not limited to generative models, pattern recognition models, and transformers, to recognize patterns between landmarks, the inventors can improve the model's training and raise detection accuracy.

Using the trained model 306, users can import medical images 308 and get the desired anatomical parameters as the output 309, as discussed in further detail below. The GUI 307 is one possible embodiment of the present technology and can be used to classify patients for providing surgical guidance.

With reference to FIGS. 3B-3F, the inventors' physics-informed deep learning approach will now be described in more detail.

As discussed above, sagittal spinopelvic balance is pivotal for comprehending spinal deformities, THA pre-operative planning, and treatment selection, but manual measurements of spinopelvic factors can be subjective and prone to error. Advanced AI and deep learning techniques offer promise in automating anatomical measure extraction from spine radiographs, potentially enhancing efficiency and accuracy.

The present system and method introduces a novel approach using bounding boxes for landmark detection, addressing the drawbacks of heatmap-based regression as highlighted above. This novel deep learning model developed by the inventors not only detects each anatomical landmark as a unique object, but also establishes a relationship graph between objects as geometrical constraints, enhancing accuracy in locating landmarks, a challenge of misdetection of adjacent landmarks reported previously in the literature. Employing this approach proves advantageous when dealing with near-identical objects, such as the femoral heads, within a single image. It significantly streamlines the process of locating these landmarks, along with adjacent spinal landmarks of a similar nature, thereby reducing overall complexity of detecting required landmarks to calculate spinopelvic measures.

As one possible embodiment, in the inventors' research, five anatomical measures were targeted to be extracted automatically in lateral X-ray images as well as two anatomical measures in AP X-ray images. The lateral measures are Sacrum Slope (SS), Pelvic Tilt (PT), Pelvic Incidence (PI), Lumbar Lordosis (LL), and Sagittal Vertical Axis (SVA). As shown in FIG. 3B, SS is the angle between the tangent line to the upper end plate of S1 (connecting line of posterior and anterior corners) and the horizontal reference line. PT is the angle between the vertical reference line and the connecting line between midpoint of sacrum upper end plate and the midpoint of connecting line between the intersection of femoral head circles (hip axis). Pelvic Incidence (PI) is a morphologic measure defined as the angle between the perpendicular line to the mid-point of the upper end plate of last vertebra of the sacrum (S1) and the connecting line of this point to the midpoint of connecting line between the intersection of femoral head circles. This measure is defined as an anatomical measure and strictly related to the shape of the pelvis. This measure mostly remains constant in each pelvic posture, and it is equal to the sum of two measures (PT and SS).

To automatically extract spinopelvic measures of interest (such as SS, PT, PI, LL, SVA, CA, and PO), the inventors adopted a landmark detection approach that treats landmarks as objects. Our method utilizes a deep learning physics-based object detection algorithm, which overcomes limitations of heatmap-based regression methods, including issues with overlapping heatmap signals and post-processing requirements. In our approach, landmarks are represented as objects with bounding boxes centered at the landmark coordinates (bx, by) and equal width (bw) and height (bh). Our labeled dataset comprises 10 classes of landmark objects (cᵢ), including the centers of femoral heads and the anterior/posterior points of S1, L1, C7 superior end plates, L5 inferior end plate, tops of the iliac crests, and any other required vertebrae to be identified in AP or lateral view. Each label includes the class number and the bounding box features Cᵢ=(cᵢ, bxᵢ, byᵢ, bwᵢ, bhᵢ).

Now referring to FIG. 3C, the deep learning model of the present system and method predicts objects with varying confidence levels, and the object with the highest confidence is used to extract the desired landmarks. To explain the rationale for considering constraints between these anatomical landmarks, the inventors can consider the scenario where a surgeon intends to annotate a lateral X-ray image in order to measure the SS. When the posterior corner of the superior sacrum end plate is identified, the surgeon can leverage the predictable relationship within the sacrum as a solid body to determine the corresponding anterior point. The inventors refer to this relationship as a geometric constraint, and the same concept has been applied for other landmarks. Imposing these constraints helps the model not only understand the features of each object (landmarks) but also enables a holistic understanding of the entire image and the interrelationships (constraints) among these landmarks. The final detected landmarks are subsequently used to calculate the desired measurements.

### Dataset and Data Preparation

In preparing the dataset, the inventors collected a total of 1,150 lateral spine X-ray images (Dataset 1, DS1) from patients referred to a hospital, between 2016 and 2022. Additionally, the inventors incorporated a dataset (Dataset 2, DS2) of 320 lateral lumbar spine and pelvic images provided by a medical company. Our datasets encompass different range of cases, including patients with hip or spine implants and images from both sitting and standing postures. Unlike some other research, the inventors included all images, even those with poor contrast or partial spine visibility. To address these issues, the inventors employed appropriate image processing filters to enhance annotation accuracy for parts with high or low intensity. By including partial spine images, our dataset enables the model to identify anatomical landmarks effectively, even in incomplete images. The utilization of two distinct datasets enabled us to evaluate the model's performance on different data sizes and imaging systems. DS1 consisted of images captured using ordinary X-ray devices, while DS2 utilized the EOS imaging system. To facilitate training, validation, and testing, the inventors divided the datasets into sets comprising 80%, 10%, and 10% of the total data, respectively. However, it will be appreciated that these percentages are just illustrative, and the datasets could be divided into data sets comprising different percentages of the total data.

A Matlab graphical user interface (GUI) was developed to facilitate image annotation (See FIG. 3C-A) In this process, 10 anatomical landmarks (superior posterior and anterior corners of S1, posterior and anterior femoral heads, inferior posterior and anterior corners of L5, superior posterior and anterior corners of L1, superior posterior and anterior corners of C7) are manually annotated in each image using the GUI. The GUI allows annotators to identify the desired points, which automatically generates corresponding bounding boxes. For accurate femoral head annotations, annotators are required to specify three points on the edge of each femoral head. The GUI then calculates and visualizes the center of the femoral heads based on the specified points. The sizes of the bounding boxes are optimized for the best mAP (i.e., maximum mean Average Precision) value to consider landmarks as objects, As an illustrative example, and not by way of limitation, the sizes of the bounding boxes could be set at 5% of the maximum image size. However, for the femoral heads, the bounding box sizes vary depending on the size of the femoral head circles. To ensure consistency, the coordinates and bounding box sizes are normalized to the maximum dimension of the image, resulting in coordinate values ranging from 0 to 1. The resulting labels are shown in FIG. 3C-A. Three expert orthopedic surgeons annotated the test dataset for benchmarking the model's accuracy. They used the developed GUI mirroring the actual software's functionalities, such as zoom and brightness adjustment. Their task was to mark images as they would pre-surgery, conducting independent validation without further guidance. The same procedure can be done for AP X-ray images to annotate required landmarks.

### Model Architecture

Heatmap-based regression has been widely used in tasks like landmark detection, despite its drawbacks such as quantization error and high computational requirements. To address these limitations and provide a more efficient alternative, the inventors introduce a novel approach called LanDet (Landmark Detection). Instead of relying on heatmaps, LanDet models individual landmarks as objects within a dense single-stage anchor-based detection framework. Furthermore, the relations between landmarks are imposed to the detection architecture as geometrical constraints. This innovative method aims to improve the efficiency and accuracy of anatomical landmark detection and clinical measurements without the need for heatmaps.

Now referring to FIG. 3D, LanDet utilizes a dense detection network denoted as DN that is trained with the multi-task loss LanDetoss. The purpose of this network is to transform an input image represented by I into a collection of output grids denoted as G. These grids contain the predicted landmark objects represented by Ôₗ. To obtain potential detection, a technique called non-maximum suppression (NMS) [60] is employed for Ôₗ. The geometrical constraints are applied on these candidate detections to generate the final predictions for Ôₗ, which are then be used to calculate desired measures.

Anchor boxes enable the model to predict more than one object in a single cell. The LanDet pipeline shown utilizes a deep convolutional neural network denoted as DN, which takes an input image I with dimensions h×w×3 and transforms it into a collection of three output grids denoted as G\hat{ }. These grids contain the predicted objects denoted as O\hat{ }. Each individual grid, denoted as Ĝₙ, has dimensions

$$\frac{h}{n} \times \frac{w}{n} \times {N}_{a} \times {N}_{o} ,$$

where n takes on values from the set {8, 16, 32}. The transformation performed by the deep network can be expressed as the following equation:

$$DN ( I ) = \hat{G}$$

where Nₐ represents the count of anchor channels, while Nₒ corresponds to the number of output channels for each object. The feature extractor DN makes effective use of Cross-Stage-Partial (CSP) bottlenecks.

Due to the properties of strided convolutions, the characteristics of an output grid cell (denoted as Ĝₙ^{i,j}) are influenced by the image patch Iₚ, defined as Iₙ_{ᵢ}_{:n}_{ᵢ₊₁}_{,n}_{ⱼ}_{:n}_{ⱼ₊₁}. Consequently, if the center of a target object (bₓ, b_{y}) lies within Iₚ, the corresponding output grid cell (Ĝₙ^{i,j}) is responsible for detecting that object. The effective range of perception of an output grid expands as n increases, implying that smaller output grids are more effective at detecting larger objects. The bounding box sizes are set to be 5% of the maximum size of the image for the landmarks on the spinal part, and for the femoral heads, the bounding box sizes are equal to the diameter of the femoral cup so that it can enclose that part.

The output grid cells (Ĝₙ^{i,j}) encompass Nₐ anchor channels, which are associated with anchor boxes Aₙ={(A_{w}_{ₐ}, Aₕ_{ₐ})}ₐ₌₁^{N}^{ₐ}. To assign a target object O to an anchor channel, a tolerance-based matching of their respective sizes is employed. This approach introduces redundancy, allowing the grid cells (Ĝₙ^{i,j}) to detect multiple objects and specialize in detecting objects of various sizes and shapes. Moreover, additional redundancy in detection is achieved by enabling the neighboring grid cells (Ĝₙ^{i±1,j}) and (Ĝₙ^{i,j±1}) to detect objects within the same image patch Iₚ.

### Loss Function

This section describes an illustrative loss function used by the inventors to train the model. However, it will be appreciated that other loss functions may be used while training a model in accordance with the present system and method.

To introduce the relations between each landmark to the model, the inventors modified the main object detection loss function to incorporate the geometric constraints as one possible embodiment of a modified loss function. A set of target grids G is created, and a multi-task loss function LanDetₗₒₛₛ is employed to train the model to learn various aspects, including the objectness \hat{p}ₒ (represented by l_{obj}), the intermediate bounding boxes \hat{t} (l_{box}), the class scores ĉ (l_{cts}), and the intermediate constraint satisfaction \hat{r} (l_{cnst}). To compute the loss components for a single image, the following procedure is followed:

$$L a n D e {t}_{l o s s} = {l}_{b o x} + {l}_{o b j} + {l}_{c l s} + {\sum }_{i = 1}^{k} {w}_{i} \cdot {l}_{cnst}$$

where k is the number of defined constraints and wᵢ are the weights for each of the constraints. the components of this loss function are defined as followed:

$${l}_{o b j} = {\sum }_{n} \frac{1}{num ( {G}_{n} )} {\sum }_{{G}_{n}} B C E ( {\hat{p}}_{o} , {p}_{o} , IoU ( \hat{t} , t ) )$$

$${l}_{b o x} = {\sum }_{n} \frac{1}{n u m ( 0 ∈ {G}_{n} )} {\sum }_{O ∈ {G}_{n}} ( 1 - IoU ( \hat{t} , t ) )$$

$${l}_{c l s} = {\sum }_{n} \frac{1}{num ( O ∈ {G}_{n} )} {\sum }_{O ∈ {G}_{n}} B C E ( \hat{c} , c )$$

$${l}_{cnst} = {\sum }_{n} \frac{1}{num ( O ∈ {G}_{n} )} {\sum }_{i ∈ {O}_{{l}_{i}}} {\sum }_{j ∈ {O}_{{l}_{j}}} {f}_{c} ( {\hat{r}}_{ij} , {r}_{i j} )$$

where BCE (binary cross-entropy), and “intersection over union” IoU (measures the overlap between the predicted bounding box and the ground truth bounding box), are crucial elements and defined as follows:

$$BCE ( c , \hat{c} ) = - ( c \cdot log ( \hat{c} ) + ( 1 - c ) \cdot log ( 1 - \hat{c} ) )$$

$$IoU = \frac{Area of Intersection}{Area of Union}$$

Furthermore, f_{c} denotes a regression-based function that characterizes the correlation among landmarks. These constraints pertain to the interrelations between landmarks on the same spinal vertebrae, as well as the Pelvic Incidence (PI) measure. As explained previously, PI is a geometric constant unique to each patient, remaining invariant even with changes in posture. For any angular constraint (e.g., PI), f_{c} represents a cosine similarity function and for the distance constraints (e.g., anterior and posterior corners of each vertebra), f_{c} represents the absolute distance loss. When Ĝₙ^{i,j,a} represents a target object O, the value of the target objectness \hat{p}ₒ is determined by multiplying it with the IoU score to encourage specialization within the anchor channel predictions. Conversely, when Ĝₙ^{i,j,a} does not represent a target object, \hat{p}ₒ is set to 0. Practical implementation involves applying the losses to a batch of images using batched grids. The total loss LanDetₗₒₛₛ is computed as a weighted sum of the loss components, scaled by the batch size n_{b}:

$$L a n D e {t}_{l o s s} = {n}_{b} ( {λ}_{b o x} \cdot {l}_{b o x} + {λ}_{o b j} \cdot {l}_{o b j} + {λ}_{c l s} \cdot {l}_{c l s} + {λ}_{cnst} . {\sum }_{i = 1}^{k} {w}_{i} \cdot {l}_{cnst} )$$

where each 1 is the weight for the corresponding loss measurement.

### Experiments

The LanDet model underwent training and testing on three distinct datasets: DS1, DS2, and their combined set. This evaluation was performed both with and without the incorporation of physics-informed constraints. The model's performance was assessed and compared against each other as well as against state-of-the-art methods.

For implementation, PyTorch 2.0 was employed, with most hyperparameters inherited and fine-tuned from. All models were trained for 300 epochs using stochastic gradient descent with Nesterov momentum, weight decay, and a learning rate decayed over a single cosine cycle, with an initial 3-epoch warm-up period. The input images were resized and padded to 640×640 while preserving the original aspect ratio. Data augmentation techniques during training encompassed mosaic, translations, horizontal flipping, and scaling. The models were trained on a Geforce RTX-4070Ti GPU with 32 GB of memory, employing batch sizes of 32. However, it will be appreciated that this is just one example of deep learning frameworks, data manipulations, and hardware that may be used.

Validation was conducted after each epoch, preserving the model weights that yielded the highest validation Average Precision (AP).

### Model Performance and Evaluation Metrics

To evaluate landmark detection as objects, mean Average Precision (mAP) was used. The calculation of mAP involves several metrics and components, including intersection over union (IOU), precision, recall, precision-recall curve, and average precision (AP). To assess the accuracy of the model predictions, the inventors employ the relative root mean square error (RRMSE) to compare the predicted values (PR) with the manual annotation labels (MA). The RRMSE is computed using the following equation:

$$RRMSE = \frac{\sqrt{\frac{1}{N} {\sum }_{i = 1}^{N} {( {y}_{i} - {v}_{i} )}^{2}}}{\sqrt{\frac{1}{N} {\sum }_{i = 1}^{N} {( {y}_{i} - \overline{y} )}^{2}}}$$

Here, yᵢ represents the manually-measured quantity the inventors aim to predict, vi denotes our model's prediction, and y⁻ is the mean of the manual annotation labels, defined as:

$$\overline{y} = \frac{1}{N} {\sum }_{i = 1}^{N} {y}_{i}$$

The RRMSE is a dimensionless metric, where a lower value indicates better accuracy (0 being the optimal value and 1 representing the threshold of uselessness. Additionally, the inventors define the detection accuracy as:

Accuracy=(1−RRMSE)×100

To evaluate the consistency among surgeons' annotations and the quality of the manual annotation labeling, the inventors involved three senior surgeons to review and annotate the test dataset. The inventors calculated the intraclass correlation coefficient (ICC) between each reviewer, as well as between the MA and PR measurements. This analysis helps us assess the level of agreement and inconsistencies in the annotations. The inventors have also evaluated the model reliability by comparing the surgeons' measurements with model prediction using the ICC metric. The ICC is a measure used to assess the reliability or agreement among surgeons, MA, and PR in this study. The inventors have used a single-rating consistency model as follows:

$$ICC = \frac{M S B S - MSWS}{M S B S + ( m - 1 ) MSWS}$$

Where;
MSBS: represents the Mean Square Between Subjects (variance between raters),
MSWS: represents the Mean Square Within Subjects (variance within raters),
m: represents the number of raters,
Using this metric, the model's reliability can be evaluated by examining the ICC value for each measure in our comparison.

### Results and Discussion

This section describes just one illustrative example of the results obtained from testing conducted by the inventors. However, it will be appreciated that this is for one possible embodiment of the system and method, and better results could be obtained by using a larger dataset, and making incremental improvements through training iterations and testing with feedback.

The performance of the LanDet model on the test datasets, which consisted of 140 images from both DS1 and DS2, was very high. The model successfully detected all landmarks in 137 images, achieving an overall detection rate of 98%. However, it encountered difficulties in two images where it failed to identify the femoral heads, and one image where the sacrum landmarks were missed. Note that during manual annotation, the annotators also faced challenges in identifying femoral heads in six test images due to obstacles or partial image cutoffs in that specific area. However, the model predicted the location of femoral heads in these challenging cases, demonstrating its robustness. Moreover, the model showed excellent performance in detecting landmarks, even in scenarios involving spinal or hip implants and low-quality images, despite the limited data available for these cases in the training and validation datasets. The accuracy of the model's landmark detection is further discussed below.

In summary, the model tested by the inventors exhibits robust performance across a range of measures, consistently matching or surpassing the literature-reported accuracy. This highlights the model's potential as an effective tool in its respective field, particularly noteworthy in its precision and reliability.

To evaluate the model reliability and to compare the model performance against surgeons, the automatic extracted measures from the model is compared to measures from three surgeon annotations. For this purpose, ICC metric is used and the results are shown in FIG. 3E. The present model showed a better consistency with surgeons compared to what has been previously reported in the literature. Mean ICC values reported in are as follows: SVA at 0.99, PT at 0.96, LL at 0.90, SS at 0.84, and PI at 0.78. The ICC values for our model and reviewers shows higher values (i.e., better consistency) in all measures. The LanDet deep learning model was able to meet the reliability of surgeon reviewers in all of the measures, as the ICC values for model versus surgeons closely mirrored those for surgeon versus surgeon, with all but three SS values (0.88, 0.89, 0.89) over the score of 0.9, indicating excellent correlation in each comparison. The inventors' findings align with the conclusions drawn in previous research, which also reported the highest error or variability and less consistency in measuring SS.

The performance of the model not only aligns well with existing literature and correlates well to surgeons' annotations but also displays exceptional proficiency in managing unique scenarios and addressing challenges associated with the identification of adjacent landmarks. This is primarily attributed to the incorporation of geometrical constraints in the LanDet physics-informed deep learning model. The inventors evaluated the model on two distinct datasets, demonstrating its adaptability to different scenarios and images from diverse sources.

Now referring to FIG. 3F, shown are four specific instances where the model accurately identified the required landmarks, even with patients having spinal or hip implants or images with partial cutoff or obstructed by protective shields. Despite the scarcity of training images for such special cases, our model proved successful in handling them effectively. It is worth noting that manually annotating landmarks in such cases, particularly those with obstacles or partial image cutoff, can be challenging.

Previous studies have highlighted the challenge of missing specific landmarks and difficulty in distinguishing between adjacent and similar anatomical landmarks. In our research, the inventors encountered similar issues until we introduced physics-informed constraints into our model.

Now referring to FIG. 3G, the concept of landmarks as objects with the integration of physics-informed constraints is shown. The primary limitation of LanDet is the availability of data. As expected, a larger training dataset would likely lead to better model performance. While the model demonstrated robustness in detecting landmarks in challenging cases, improving the accuracy of these detections would be possible by increasing the number of images from patients with abnormalities or spinal implants in the dataset.

In the above illustrative embodiment, the inventors presented a novel deep learning approach for detecting anatomical landmarks as objects, surpassing the limitations of previous models that heavily relied on heat-map regression. By incorporating physics-informed constraints into our deep learning models, we achieved significant improvements in landmark detection accuracy. Moreover, the approach demonstrated robustness in challenging scenarios, including cases with implants, protected regions, and partially obscured images, even when training data for such scenarios was limited. Furthermore, the model effectively addressed the issue of mis-detection of similar or adjacent landmarks. The landmark detection performance for SS, PT, PI, LL, and SVA measures was evaluated, comparing results between datasets of different sizes and against the existing literature. The model achieved competitive performance while offering the aforementioned advantages. To assess the reliability of our model, we compared its predictions against those of three senior surgeons, using the ICC metric. The results revealed a high level of agreement between our model and the expert surgeons.

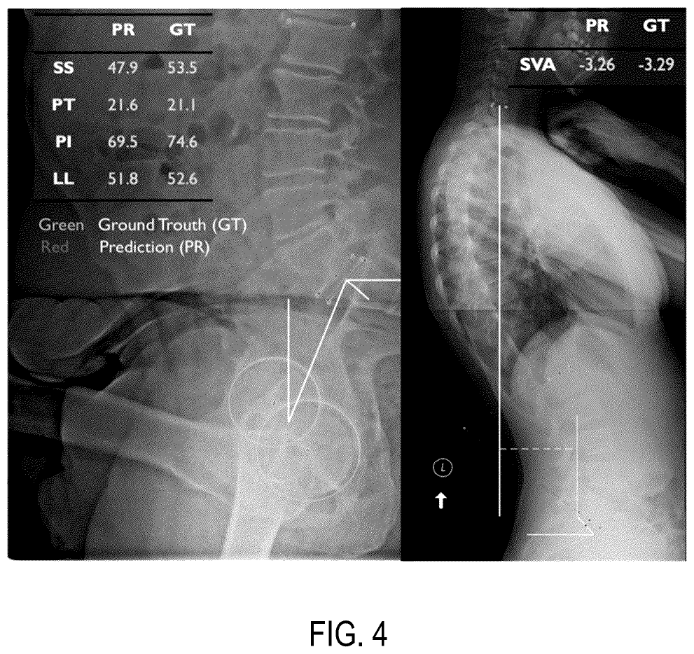

By applying the above approach of automated detection of landmark features, FIG. 4 shows a possible output of the model (anatomical parameters) of a lateral view X-ray image presented to the user comparing with ground truth manual annotation.

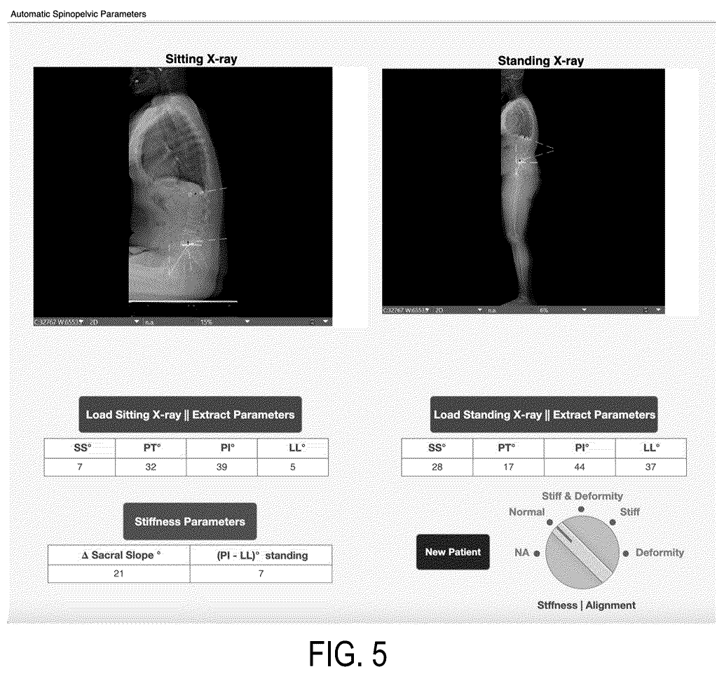

FIG. 5 shows a graphical user interface (GUI) in which the invention has been used to categorize patients based on different spinal stiffnesses. The developed model 306 and the computing device 3 can be coded so that the user is able to interactively provide feedback (relocated landmarks) to the model, which updates the hyper-parameters and improves future predictions.

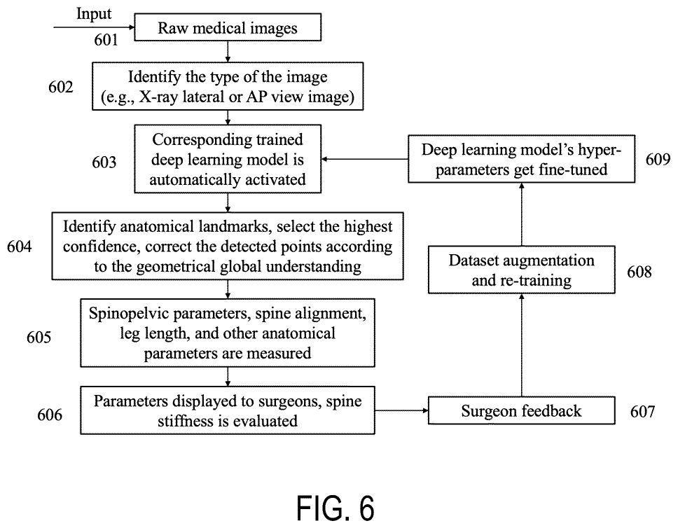

In FIG. 6, a flow diagram shows how the present invention may be used for automatic extraction of anatomical parameters and providing surgical guidance according to patient categorization. At 601 the user uploads a medical image 201 to the computer system and specifics the type of the imported image 602. The computing device 3 then activates the corresponding trained deep learning model 406 and uses optimized hyper-parameters to perform various image processing tasks to identify anatomical landmarks 604. Using the detected landmarks, the computer system can measure anatomical parameters 605 and display them to the user 606. The users can update the detected landmarks if the detection does not satisfy their requirements (e.g., any possible error with the detected landmarks according to their understanding). The provided feedback 607 will be recorded and is fed into the model again to augment the dataset 608 and update the model's hyper-parameters 609. The updated model will perform better detection because of the improvements in the dataset and trained model.

While the above illustrative examples have focused on a lateral view of a patient's spine, it will be appreciated that the physics-based approach can be extended to other views, and to various different parts of the musculoskeletal system as well. For example, in FIG. 7, shown is an anterior-posterior view drawing indicates the anatomical parameters and required landmarks to be detected to measure the parameters from “medical images”. In the schematic AP image 7, part 701 represents how the technology identifies each vertebra as a single object and detects the corners of that vertebra, 701SL, 701SR, 701BL, and 701BR, which are required for spinopelvic parameter extraction. Part 702 represents the normal pelvis, 703 represents the pelvic obliquity, part 704 represents the horizontal reference line, part 705 represents vertical reference line. The trained deep learning model 306 can detect the tops of the iliac crests, 706A and 706B, and by drawing a connecting line 706C between them, the degree of obliquity can be assessed by measuring the angle between 704 and 706C, or between 705 and 707 (perpendicular to 706C). The trained model 306 detects and the top of the lowest tilted vertebra 708 and the bottom of the uppermost tilted vertebra 709 in the curve (or the bottom of the lowest tilted vertebra 708 and the top of the uppermost tilted vertebra 709, not depicted here). To assess the severity of scoliosis, the model detects the corners of 708 and 709. The Cobb angle 710, a critical parameter for scoliosis assessment, is formed at the intersection of lines connecting points 708A to 708B and 709A to 709B. Apical Vertebral Translation (AVT) is another parameter that can be measured. This measures the lateral displacement of the apical vertebra 711 (the most rotated and laterally deviated vertebra in the curve) from the midline vertical reference or central sacral vertical line (CSVL) 705.

An analogous approach may be used to automatically detect anatomical landmarks using a physics-based approach, and by measuring geometric relations between the anatomical landmarks expressed as objects in the plurality of medical images to establish geometric constraints between the objects. An analogous approach can also be used to retrain the deep learning model to automatically detect anatomical landmarks expressed as objects in new medical images by specifying expected locations of one or more objects based on the established geometric constraints.

Advantageously, the current system and method presents a significant advancement in the field of anatomical landmark detection utilizing deep learning techniques. Its success in handling challenging scenarios and achieving comparable performance to expert evaluations makes it a valuable tool for real world clinical and surgical applications.

Thus, in an aspect, there is provided a computer-implemented method for automatic detection and measurement of anatomical landmarks, the method executable on a computing device having a processor and a memory, comprising: (i) providing a deep learning model trained on a training dataset of manually annotated anatomical landmarks in a plurality of medical images; (ii) implementing a physics-informed approach by measuring geometric relations between the anatomical landmarks expressed as objects in the plurality of medical images to establish geometric constraints between the objects; and (iii) retraining the deep learning model to automatically detect anatomical landmarks expressed as objects in new medical images by specifying expected locations of one or more objects based on the established geometric constraints.

In an embodiment, the medical images comprise one or more of lateral X-rays, AP X-rays, CT-scans, MRI, and Ultrasound images.

In another embodiment, the deep learning model is trained for diagnosing skeletal disorders, and planning surgical procedures to address the skeletal disorders.

In another embodiment, the skeletal disorders are directed to a patient's spine, and the surgical procedure comprises spine surgery.

In another embodiment, the model is further developed by utilizing the measured geometric relations to determine the severity of a skeletal disorder.

In another embodiment, the model is further developed for virtual fitting and sizing of a surgical implant based on the geometric constraints prior to surgery.

In another embodiment, the method further comprises storing any manual alternations made to the automatically detected anatomical landmarks in an augmented dataset for retraining the deep learning model.

In another aspect, there is provided a system for automatic detection and measurement of anatomical landmarks, the system including a processor and a memory, and adapted to: (i) utilize the processor, providing a deep learning model trained on a training dataset of manually annotated anatomical landmarks in a plurality of medical images; (ii) implement a physics-informed approach by measuring geometric relations between the anatomical landmarks expressed as objects in the plurality of medical images to establish geometric constraints between the objects; and (iii) retrain the deep learning model to automatically detect anatomical landmarks expressed as objects in new medical images by specifying expected locations of one or more objects based on the established geometric constraints.

In an embodiment, the medical images comprise one or more of lateral X-rays, AP X-rays, CT-scans, MRI, and Ultrasound images.

In another embodiment, the deep learning model is trained for diagnosing skeletal disorders, and planning surgical procedures to address the skeletal disorders.

In another embodiment, the skeletal disorders are directed to a patient's spine, and the surgical procedure comprises spine surgery.

In another embodiment, the model is further developed by utilizing the measured geometric relations to determine the severity of a skeletal disorder.

In another embodiment, the model is further developed for virtual fitting and sizing of a surgical implant based on the geometric constraints prior to surgery.

In another embodiment, the system further comprises storing any manual alternations made to the automatically detected anatomical landmarks in an augmented dataset for retraining the deep learning model.

In another aspect, there is provided a non-transitory computer-readable storage medium having stored thereon instructions, which when executed by one or more processors, causes the processors to perform operations comprising: (i) providing a deep learning model trained on a training dataset of manually annotated anatomical landmarks in a plurality of medical images; (ii) implementing a physics-informed approach by measuring geometric relations between the anatomical landmarks expressed as objects in the plurality of medical images to establish geometric constraints between the objects; and (iii) retraining the deep learning model to automatically detect anatomical landmarks expressed as objects in new medical images by specifying expected locations of one or more objects based on the established geometric constraints.

In an embodiment, the medical images comprise one or more of lateral X-rays, AP X-rays, CT-scans, MRI, and Ultrasound images.

In another embodiment, the deep learning model is trained for diagnosing skeletal disorders, and planning surgical procedures to address the skeletal disorders.

In another embodiment, the skeletal disorders are directed to a patient's spine, and the surgical procedure comprises spine surgery.

In another embodiment, the model is further developed by utilizing the measured geometric relations to determine the severity of a skeletal disorder.

In another embodiment, the model is further developed for virtual fitting and sizing of a surgical implant based on the geometric constraints prior to surgery.

While various illustrative embodiments have been described above, it will be appreciated that the scope of the invention is defined by the following claims.

1. A computer-implemented method for automatic detection and measurement of anatomical landmarks, the method executable on a computing device having a processor and a memory, comprising:
(i) providing a deep learning model trained on a training dataset of manually annotated anatomical landmarks in a plurality of medical images;
(ii) implementing a physics-informed approach by measuring geometric relations between the anatomical landmarks expressed as objects in the plurality of medical images to establish geometric constraints between the objects; and
(iii) retraining the deep learning model to automatically detect anatomical landmarks expressed as objects in new medical images by specifying expected locations of one or more objects based on the established geometric constraints.

2. The computer-implemented method of claim 1, wherein the medical images comprise one or more of lateral X-rays, AP X-rays, CT-scans, MRI, and Ultrasound images.

3. The computer-implemented method of claim 1, wherein the deep learning model is trained for diagnosing skeletal disorders, and planning surgical procedures to address the skeletal disorders.

4. The computer-implemented method of claim 3, wherein the skeletal disorders are directed to a patient's spine, and the surgical procedure comprises spine surgery.

5. The computer-implemented method of claim 1, wherein the model is further developed by utilizing the measured geometric relations to determine the severity of a skeletal disorder.

6. The computer-implemented method of claim 1, wherein the model is further developed for virtual fitting and sizing of a surgical implant based on the geometric constraints prior to surgery.

7. The computer-implemented method of claim 1, further comprising storing any manual alternations made to the automatically detected anatomical landmarks in an augmented dataset for retraining the deep learning model.

8. A system for automatic detection and measurement of anatomical landmarks, the system including a processor and a memory, and adapted to:
(i) utilize the processor, providing a deep learning model trained on a training dataset of manually annotated anatomical landmarks in a plurality of medical images;
(ii) implement a physics-informed approach by measuring geometric relations between the anatomical landmarks expressed as objects in the plurality of medical images to establish geometric constraints between the objects; and
(iii) retrain the deep learning model to automatically detect anatomical landmarks expressed as objects in new medical images by specifying expected locations of one or more objects based on the established geometric constraints.

9. The system of claim 8, wherein the medical images comprise one or more of lateral X-rays, AP X-rays, CT-scans, MRI, and Ultrasound images.

10. The system of claim 8, wherein the deep learning model is trained for diagnosing skeletal disorders, and planning surgical procedures to address the skeletal disorders.

11. The system of claim 10, wherein the skeletal disorders are directed to a patient's spine, and the surgical procedure comprises spine surgery.

12. The system of claim 8, wherein the model is further developed by utilizing the measured geometric relations to determine the severity of a skeletal disorder.

13. The system of claim 8, wherein the model is further developed for virtual fitting and sizing of a surgical implant based on the geometric constraints prior to surgery.

14. The system of claim 8, further comprising storing any manual alternations made to the automatically detected anatomical landmarks in an augmented dataset for retraining the deep learning model.

15. A non-transitory computer-readable storage medium having stored thereon instructions, which when executed by one or more processors, causes the processors to perform operations comprising:
(i) providing a deep learning model trained on a training dataset of manually annotated anatomical landmarks in a plurality of medical images;
(ii) implementing a physics-informed approach by measuring geometric relations between the anatomical landmarks expressed as objects in the plurality of medical images to establish geometric constraints between the objects; and
(iii) retraining the deep learning model to automatically detect anatomical landmarks expressed as objects in new medical images by specifying expected locations of one or more objects based on the established geometric constraints.

16. The non-transitory computer-readable storage medium of claim 15, wherein the medical images comprise one or more of lateral X-rays, AP X-rays, CT-scans, MRI, and Ultrasound images.

17. The non-transitory computer-readable storage medium of claim 15, wherein the deep learning model is trained for diagnosing skeletal disorders, and planning surgical procedures to address the skeletal disorders.

18. The non-transitory computer-readable storage medium of claim 17, wherein the skeletal disorders are directed to a patient's spine, and the surgical procedure comprises spine surgery.

19. The non-transitory computer-readable storage medium of claim 15, wherein the model is further developed by utilizing the measured geometric relations to determine the severity of a skeletal disorder.

20. The non-transitory computer-readable storage medium of claim 15, wherein the model is further developed for virtual fitting and sizing of a surgical implant based on the geometric constraints prior to surgery.
